(19) United States
(12) Patent Application Publication
(10) Pub. No.: US 2022/0348640 A1
(43) Pub. Date: Nov. 3, 2022

(54) ELUTION OF MONOCLONAL ANTIBODIES IN PROTEIN A AFFINITY CHROMATOGRAPHY

(71) Applicant: Merck Patent GmbH, Darmstadt (DE)

(72) Inventors: Christoph KORPUS, Darmstadt (DE); Supriyadi HAFIZ, Darmstadt (DE)

(73) Assignee: Merck Patent GmbH, Darmstadt (DE)

(21) Appl. No.: 17/765,043

(22) PCT Filed: Oct. 1, 2020
(86) PCT No.: PCT/EP2020/077445

§ 371 Date: Mar. 30, 2022

(30) Foreign Application Priority Data
Oct. 4, 2019 (EP) 19201486.8

(51) Int. Cl. C07K 16/00; C07K 1/22

(52) U.S. Cl. CPC C07K 16/00; C07K 1/22

(57) ABSTRACT

The invention provides a method for eluting a monoclonal antibody from a Protein A affinity chromatography column to which the monoclonal antibody is bound comprising
a) contacting the affinity chromatography column with an elution buffer comprising a poly (ethylene glycol) polymer;
b) collecting one or more fractions containing the monoclonal antibody obtained from step (a)
c) combining the fractions obtained from step (b) to form an elution product pool.

### TECHNICAL FIELD

The present invention relates to an improved method for eluting a monoclonal antibody (mAb) from a Protein A affinity chromatography column to which the monoclonal antibody is bound.

### STATE OF THE ART

The therapeutic applications for monoclonal antibodies (mAbs) play an increasing role in today's medical needs. Protein A affinity chromatography is a well-known and widely-used tool for purifying monoclonal antibodies. Due to its specific interaction with the antibody, which take place between the Fc region of mAbs and immobilized protein A, and its ability to tolerate high conductivities, Protein-A chromatography allows direct loading of harvested cell culture fluid (HCCF) and enables removal of a vast majority of process and product related impurities while enriching the antibody pool (Vunnum et al., 2009. Protein-A based affinity chromatography. In: Gottschalk U, editor. Process scale purification of antibodies. Hoboken, N.J.: John Wiley BT & Sons, Inc. p 79-102).

The downstream processing of biotechnologically produced monoclonal antibodies typically comprises at least two chromatography steps: a first affinity chromatography step using e.g. Protein A, to remove non-antibody molecules from the harvested cell culture fluid (HCCF), followed by one or more further steps, such as ion exchange chromatography steps. The elution of the monoclonal antibodies from the chromatography column is an essential step in process performance, which determines separation selectivity and the pool volume containing the product (Angelo, J. M., Lenhoff, A. M. 2016. Determinants of protein elution rates from preparative ion-exchange adsorbents, J Chromatogr A; 1440; 94-104). Not only with regard to pharmaceutical formulations but also with regard to intermediates in downstream processing, concentrated solutions are required in order to achieve low volumes for economic handling. Therefore, large pool volumes and slow elution rates are undesirable for affinity-based separations such as Protein A chromatography. Hence, there is a significant interest in improvements in the downstream process particularly with respect to higher capacity and reduced processing time.

### SUMMARY OF THE INVENTION

It was surprisingly found that the addition of a poly (ethylene glycol) polymer to the elution buffer in Protein A chromatography of monoclonal antibodies leads to enhanced antibody elution resulting in significantly lower elution pool volumes while maintaining a comparable yield compared to control conditions without the addition of an excipient or control conditions with the addition of selected disaccharides or polyols that are commonly used for formulation applications to stabilize proteins and prevent aggregation.

In particular, the invention provides a method for eluting a monoclonal antibody from a Protein A affinity chromatography column to which the monoclonal antibody is bound comprising

a) bringing the affinity chromatography column into contact with an elution buffer comprising a poly (ethylene glycol) polymer;

b) collecting one or more fractions containing the monoclonal antibody obtained from step (a)

c) potentially combining the fractions obtained from step (b) to form an elution product pool.

Advantageously, the separation under these conditions can be carried out using much lower amounts of eluent.

At the same time, the product can be obtained in higher yield and with improved purity. The examples given below show this very clearly.

It was found that preferably, the elution buffer has a concentration of poly (ethylene glycol) polymer from 2% to 15% by weight, more preferably from 5% to 10% by weight.

In a preferred embodiment of the invention, the poly (ethylene glycol) polymer has an average molecular weight in the range of 1,000 g/mol to 10,000 g/mol, more preferred from 3,000 g/mol to 5,000 g/mol.

In another preferred embodiment of the invention the elution buffer is a citrate buffer.

According to an advantageous aspect of the invention the elution step (a) comprises contacting the affinity chromatography column with the elution buffer using an elution buffer gradient from pH 5.5 to pH 2.75.

In a further preferred embodiment of the invention the elution product pool has a pH from about 3.9 to about 4.2.

### DETAILED DESCRIPTION OF THE INVENTION

In downstream processing of monoclonal antibodies it is desirable for affinity-based chromatography processes such as Protein A chromatography to obtain concentrated solutions with low eluate pool volumes. The present invention now provides a method for eluting a monoclonal antibody from a Protein A affinity chromatography column comprising the use of an elution buffer comprising a poly (ethylene glycol) polymer. The addition of poly (ethylene glycol) to the elution buffer was found to shift the elution to lower pH values while showing a sharp elution peak. This allows collecting highly concentrated eluate fractions from the column outlet and a reduced overall eluate pool volume. Furthermore, a low pH following elution is beneficial for viral inactivation. For the down stream processing of monoclonal antibodies this improvement results in higher capacity and reduced processing time.

As used herein, the term “antibody” refers to any form of antibody or fragment thereof that exhibits the desired biological activity. Thus, it is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. “Isolated antibody” refers to the purification status of a binding compound and in such context means the molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

The term “monoclonal antibody”, as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al., (1991) Nature 352: 624-628 and Marks et al., (1991) J. Mol. Biol. 222: 581-597, for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., (1984) Proc. Natl. Acad. Sci. USA 81: 6851-6855).

The term “Protein A affinity chromatography” shall refer to the separation or purification of substances and/or particles using protein A, where the protein A is generally immobilized on a solid phase. Protein A is a 40-60 kD cell wall protein originally found in Staphylococcus aureus. The binding of antibodies to protein A resin is highly specific. Protein A affinity chromatography columns for use in protein A affinity chromatography herein include, but are not limited to, Protein A immobilized on a polyvinylether solid phase, e.g. the Eshmuno® columns (Merck, Darmstadt, Germany), Protein A immobilized on a pore glass matrix, e.g. the ProSep® columns (Merck, Darmstadt, Germany) Protein A immobilized on an agarose solid phase, for instance the MABSELECT™ SuRe™ columns (GE Healthcare, Uppsala, Sweden).

The term “buffer” as used herein shall refer to a buffered solution that resists changes in pH by the action of its acid-base conjugate components. The “elution buffer” is the buffer which is used to elute a protein from the chromatography column. The elution buffer for the Protein A affinity chromatography of this invention has a pH in a range of about 2.75 to 5.5. Examples of buffers that will control the pH within this range include phosphate, acetate, citrate or ammonium buffers, or more than one. The preferred such buffer is citrate.

The terms “poly (ethylene glycol)” or “PEG” shall refer to long chain, linear synthetic polymers composed of ethylene oxide units. The ethylene oxide units can vary such that PEG compounds can be obtained with molecular weights ranging from approximately 200 g/mol to 100,000 g/mol. Such poly (ethylene glycol) may contain one or more further chemical group(s), which are necessary for binding reactions, which results from the chemical synthesis of the molecule, or which is a spacer for optimal distance of parts of the molecule. These further chemical groups are not used for the calculation of the molecular weight of the poly (ethylene glycol). In addition, such a poly (ethylene glycol) may comprise one or more poly (ethylene glycol)-chains, which are linked together. A poly (ethylene glycol) with more than one poly (ethylene glycol)-chain is called multiarmed or branched poly (ethylene glycol).

Although poly (ethylene glycols) vary substantially by molecular weight, polymers having molecular weights ranges from about 400 g/mol to about 30,000 g/mol are usually suitable. In the examples of the invention, polyethylene glycol of an average molecular weight of 4,000 g/mol (PEG4000) is selected. In preferred embodiments of the invention, polyethylene glycols having an average molecular weight in the range of 1,000 g/mol to 10,000 g/mol, more preferred from 3,000 g/mol to 5,000 g/mol are suitably selected.

The examples described below therefore provide a method of separating a monoclonal antibody from a Protein A affinity chromatography column to which the monoclonal antibody is bound by use of a reduced volume of eluent comprising the steps of:

a) bringing the loaded affinity chromatography column into contact with an elution buffer comprising a poly (ethylene glycol) polymer and inducing a sharp, narrow elution peak,

b) collecting one or more of the eluted fractions containing the monoclonal antibody obtained from step (a),

c) potentially combining the fractions obtained from step (b) to form an elution product pool,

whereby

d) an improved purity of the desired monoclonal antibody is achieved and about 9% and more of HCP is separated off and an increase in yield by more than 4% is achieved.

Advantageously by carrying out this method the demand for eluent is reduced by about at least 11% by volume.

The following examples show that by optimizing the amount and the selection of the added excipient, the required amounts of eluent can still be reduced, whereby the achievable purity can also be improved.

### BRIEF DESCRIPTION OF THE FIGURES

FIG. 1 shows clarified harvest mAbA elution profiles on Eshmuno® A using pH gradient elution with and without addition of excipients (Example 2)

FIG. 2 shows clarified harvest mAbA elution profiles on ProSep® Ultra Plus using pH gradient elution with and without addition of excipients (Example 3).

FIG. 3 shows clarified harvest mAbA elution profiles on MabSelect™ Sure™ using pH gradient elution with and without addition of excipients (Example 4).

FIG. 4 shows clarified harvest mAbB elution profiles on Eshmuno® A using pH gradient elution with and without addition of excipients (Example 5).

FIG. 5 shows clarified harvest mAbB elution profiles on ProSep® Ultra Plus using pH gradient elution with and without addition of excipients (Example 6)

FIG. 6 shows clarified harvest mAbB elution profiles on MabSelect™ SuRe™ using pH gradient elution with and without addition of excipients (Example 7).

FIG. 7 shows host cell protein (HCP) profile of mAbB in different conditions (with or without additives) on Eshmuno® A during pH gradient elution (Example 5).

FIG. 8 shows the purity of elution pool based on comparison of HCP content of pooled fractions with total HCP content during pH gradient elution on Eshmuno® A (Example 5).

FIG. 9 shows host cell protein (HCP) profile of mAbB in different conditions (with or without additives) on ProSep® Ultra Plus during pH gradient elution (Example 6).

FIG. 10 shows the purity of elution pool based on comparison of HCP content of pooled fractions with total HCP content during pH gradient elution on ProSep® Ultra Plus (Example 6).

FIG. 11 shows host cell protein (HCP) profile of mAbB in different conditions (with or without additives) on MabSelect™ SuRe™ during pH gradient elution (Example 7).

FIG. 12 shows the purity of elution pool based on comparison of HCP content of pooled fractions with total HCP content during pH gradient elution on MabSelect™ SuRe™ (Example 7).

The present description enables the person skilled in the art to apply the invention comprehensively. Even without further comments, it is assumed that a person skilled in the art will be able to utilise the above description in the broadest scope.

Practitioners will be able, with routine laboratory work, using the teachings herein, to separate proteins as defined above efficiently in the new process. If anything is still unclear, it is understood that the publications and patent literature cited should be consulted. Accordingly, these documents are regarded as part of the disclosure content of the present description.

For better understanding and in order to illustrate the invention, examples are given below which are within the scope of protection of the present invention. These examples also serve to illustrate possible variants. Owing to the general validity of the inventive principle described, however, the examples are not suitable for reducing the scope of protection of the present application to these alone.

Furthermore, it goes without saying to the person skilled in the art that, both in the examples given and also in the remainder of the description, the component amounts present in the compositions always only add up to 100% by weight, volume or mol-%, based on the composition as a whole, and cannot exceed this, even if higher values could arise from the percent ranges indicated. Unless indicated otherwise, % data are % by weight, volume or mol-%, with the exception of ratios, which are shown in volume data, such as, for example, eluents, for the preparation of which solvents in certain volume ratios are used in a mixture.

The temperatures given in the examples and the description as well as in the claims are always in ° C.

### EXAMPLES

### Example 1: Preparation of Buffer and Excipient Solutions

All buffers and excipients were filtered using a 0.45 μm HAWP mixed cellulose ester filter (Merck, Darmstadt, Germany) and degassed for 20 min in an ultrasonic bath before use. For all Protein A chromatography runs the following buffers were prepared and used:

TABLE 1
Buffer A1 for Protein A chromatography pH 5.50
Buffer A1, pH 5.50

| | Concentration | Concentration |
|---|---|---|
| Substance | [mmol/L] | [g/L] |
| Citric acid*1H₂O | 100.0 | 21.01 |
| NaOH | 255.0 | Titrated until pH 5.50 |

TABLE 2
Buffer A2 for Protein A chromatography pH 7.00
Buffer A2, pH 7.00

| | Concentration | Concentration |
|---|---|---|
| Substance | [mmol/L] | [g/L] |
| Citric acid*1H₂O | 100.0 | 21.01 |
| NaOH | 298.0 | Titrated until pH 7.00 |

TABLE 3
Buffer B for Protein A chromatography pH 2.75
Buffer B, pH 2.75

| | Concentration | Concentration |
|---|---|---|
| Substance | [mmol/L] | [g/L] |
| Citric acid*1H₂O | 100.0 | 21.01 |
| NaOH | 33.0 | Titrated until pH 2.75 |

The following excipients were chosen based on their ability to protect antibodies from aggregation:

TABLE 4
Applied excipients with applied concentrations,
manufacturer and quality standard

| Substance | Applied concentrations | Manufacturer/Quality |
|---|---|---|
| Sucrose | 0.5M | Merck/Emprove ® |
| Sorbitol | 0.5M | Merck/Emprove ® |
| Mannitol | 0.5M | Merck/Emprove ® |
| Trehalose | 0.5M | Merck/For Biochemistry |
| PEG4000 | 5% (w/w) | Merck/Emprove ® |

### Example 1.1: Preparation of 0.5M Sucrose in Citrate Buffer pH 5.5

171.1 g Sucrose (M=342.29 g/mol) was weighed into an appropriate flask. Approximately 800 ml 0.1M Na-Citrate buffer pH 5.5 was added and the solution was stirred until the substance was completely dissolved. The pH was adjusted to 5.5+/−0.05 using 1M HCl. Afterwards, the solution was transferred to a 1000.0 ml volumetric graduated flask and filled to the mark with 0.1M Na-Citrate buffer pH 5.5 and mixed thoroughly.

### Example 1.2: Preparation of 0.5M Sucrose in Citrate Buffer pH 2.75

171.1 g Sucrose (M=342.29 g/mol) was weighed into an appropriate flask. Approximately 800 ml 0.1M Na-Citrate buffer pH 2.75 was added and the solution was stirred until the substance was completely dissolved. The pH was adjusted to 2.75+/−0.05 using 1M HCl. Afterwards, the solution was transferred to a 1000.0 ml volumetric graduated flask and filled to the mark with 0.1M Na-Citrate buffer pH 2.75 and mixed thoroughly.

### Example 1.3: Preparation of 0.5M Trehalose in Citrate Buffer pH 5.5

171.1 g Trehalose (M=342.29 g/mol) was weighed into an appropriate flask. Approximately 800 ml 0.1M Na-Citrate buffer pH 5.5 was added and the solution was stirred until the substance was completely dissolved. The pH was adjusted to 5.5+/−0.05 using 1M HCl. Afterwards, the solution was transferred to a 1000.0 ml volumetric graduated flask and filled to the mark with 0.1M Na-Citrate buffer pH 5.5 and mixed thoroughly.

### Example 1.4: Preparation of 0.5M Trehalose in Citrate Buffer pH 2.75

171.1 g Trehalose (M=342.29 g/mol) was weighed into an appropriate flask. Approximately 800 ml 0.1M Na-Citrate buffer pH 2.75 was added and the solution was stirred until the substance was completely dissolved. The pH was adjusted to 2.75+/−0.05 using 1M HCl. Afterwards, the solution was transferred to a 1000.0 ml volumetric graduated flask and filled to the mark with 0.1M Na-Citrate buffer pH 2.75 and mixed thoroughly.

### Example 1.5: Preparation of 0.5M Mannitol in Citrate Buffer pH 5.5

91.09 g Mannitol (M=182.17 g/mol) was weighed into an appropriate flask. Approximately 800 ml 0.1M Na-Citrate buffer pH 5.5 was added and the solution was stirred until the substance was completely dissolved. The pH was adjusted to 5.5+/−0.05 using 1M HCl. Afterwards, the solution was transferred to a 1000.0 ml volumetric graduated flask and filled to the mark with 0.1M Na-Citrate buffer pH 5.5 and mixed thoroughly.

### Example 1.6: Preparation of 0.5M Mannitol in Citrate Buffer pH 2.75

91.09 g Mannitol (M=182.17 g/mol) was weighed into an appropriate flask. Approximately 800 ml 0.1M Na-Citrate buffer pH 2.75 was added and the solution was stirred until the substance was completely dissolved. The pH was adjusted to 2.75+/−0.05 using 1M HCl. Afterwards, the solution was transferred to a 1000.0 ml volumetric graduated flask and filled to the mark with 0.1M Na-Citrate buffer pH 2.75 and mixed thoroughly.

### Example 1.7: Preparation of 0.5M Sorbitol in Citrate Buffer pH 5.5

91.09 g Sorbitol (M=182.17 g/mol) was weighed into an appropriate flask. Approximately 800 ml 0.1M Na-Citrate buffer pH 5.5 was added and the solution was stirred until the substance was completely dissolved. The pH was adjusted to 5.5+/−0.05 using 1M HCl. Afterwards, the solution was transferred to a 1000.0 ml volumetric graduated flask and filled to the mark with 0.1M Na-Citrate buffer pH 5.5 and mixed thoroughly.

### Example 1.8: Preparation of 0.5M Sorbitol in Citrate Buffer pH 2.75

91.09 g Sorbitol (M=182.17 g/mol) was weighed into an appropriate flask. Approximately 800 ml 0.1M Na-Citrate buffer pH 2.75 was added and the solution was stirred until the substance was completely dissolved. The pH was adjusted to 2.75+/−0.05 using 1M HCl. Afterwards, the solution was transferred to a 1000.0 ml volumetric graduated flask and filled to the mark with 0.1M Na-Citrate buffer pH 2.75 and mixed thoroughly.

### Example 1.9: Preparation of 5% (w/v) PEG4000 in Citrate Buffer pH 5.5

50 g PEG4000 (M=3500-4500 g/mol) was weighed into an appropriate flask. Approximately 800 ml 0.1M Na-Citrate buffer pH 5.5 was added and the solution was stirred until the substance was completely dissolved. The pH was adjusted to 5.5+/−0.05 using 1M HCl. Afterwards, the solution was transferred to a 1000.0 ml volumetric graduated flask and filled to the mark with 0.1M Na-Citrate buffer pH 5.5 and mixed thoroughly.

### Example 1.10: Preparation of 5% (w/v) PEG4000 in Citrate Buffer pH 2.75

50 g PEG4000 (M=3500-4500 g/mol) was weighed into an appropriate flask. Approximately 800 ml 0.1M Na-Citrate buffer pH 2.75 was added and the solution was stirred until the substance was completely dissolved. The pH was adjusted to 2.75+/−0.05 using 1M HCl. Afterwards, the solution was transferred to a 1000.0 ml volumetric graduated flask and filled to the mark with 0.1M Na-Citrate buffer pH 2.75 and mixed thoroughly.

### Example 2: Elution Performance of Clarified Harvest mAbA on Eshmuno A

### Protein A Chromatography Resin:

The Eshmuno® base material is a rigid and hydrophilic polymer based on polyvinylether. Immobilized onto it is the C domain of Staphylococcus aureus Protein A in a pentameric form, which is recombinantly produced in E. coli. Eshmuno® A is from Merck (Darmstadt, Germany) and the column was packed by Repligen GmbH (Ravensburg, Germany).

TABLE 5
Column parameters for applied Eshmuno ® A resin

| | | |
|---|---|---|
| Column length | 2 | cm |
| Column inner diameter | 0.8 | cm |
| Column volume | 1 | mL |
| Mean particle diameter | ~50 | μm |

| | |
|---|---|
| Base material | hydrophilic polyvinylether |
| Functional group | recombinant Protein A produced in E. coli, |
| | derived from C domain of native Protein A |
| Lot # | K93457960 |
| Serial # | 00168 |

### Antibody Sample Preparation:

The model antibody mAbA is a monoclonal antibody (Approximately 152 kDa) with a pl˜7.01-8.58. It was used as clarified cell culture harvest, which was filtrated using a VacuCap® 90 PF Filter Unit with 0.8/0.2 μm Supor® membrane (Pall Corporation, NY, USA). The solution has a concentration of 0.943 mg/mL, a pH of 7.0 and a conductivity of 12 mS/cm.

### Protein A Chromatography Method:

The Protein A chromatography was done using the following method parameters:

TABLE 6
Method parameters for Protein A Chromatography

| Column | Eshmuno ® A | |
|---|---|---|
| System | Äkta purifier | |
| Flow | 120 cm/h (1 mL/min) | |
| Sample | mAbA HCCF | |
| Sample load | 30 | mg/mL |
| Fractionation | 2 | mL |
| Buffers | 0.1M Citric acid, pH 7; 0.1M Citric acid, pH 5.5 and pH | |
| | 2.75 with added sucrose, trehalose, mannitol, sorbitol | |
| | or PEG4000 as described in Example 1 | |
| Equilibration | 10 | CV |
| Inject | 20.69 | CV |
| Wash pH 7 | 5 | CV |
| Wash pH 5.5 | 10 CV (pooled as 2 × 5 CV) | |
| Gradient | 30 | CV |
| 100% B | 5 | CV |

Six elution runs were carried out with each of the five excipient containing elution buffers according to Example 1 and with elution buffer containing no excipient as a reference. Elution was carried out at a defined gradient slope by applying a linear gradient of 30 CV from pH 5.5 to pH 2.75. Fractions of elution peaks were collected between 30 mAU of start peak signal and 30 mAU of end peak signal at UV 280 nm (2 mm path length) from different chromatography runs and analyzed for pH, volume, yield* and HCP content* (*only applied on mAbB).

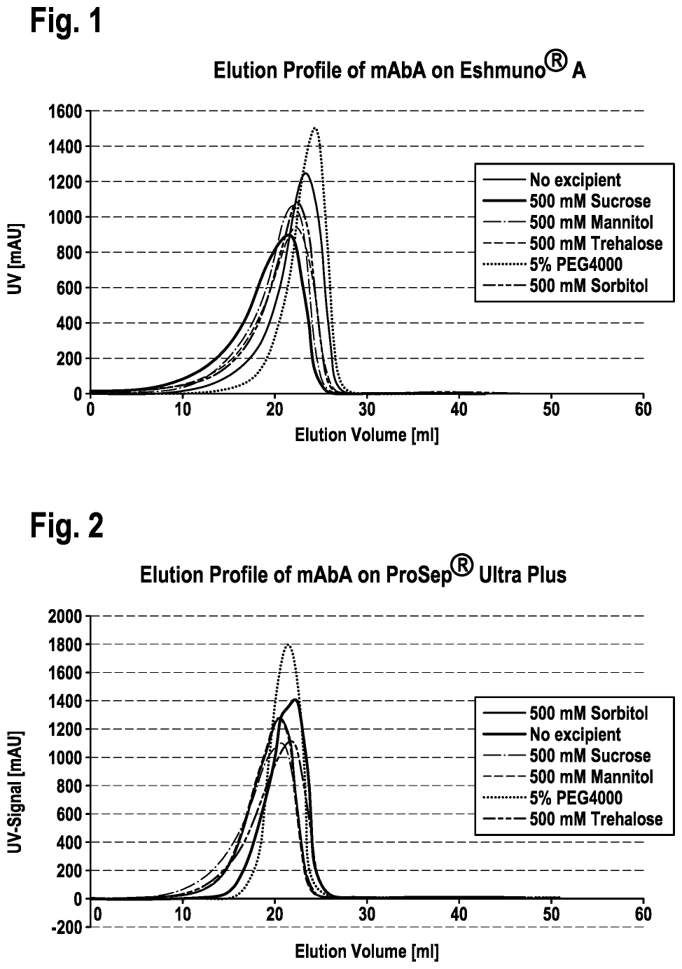

The following results were obtained and are represented graphically as elution profiles in FIG. 1:

TABLE 7
Elution performance of clarified harvest mAbA on Eshmuno A

| | pH-Value | | | |
|---|---|---|---|---|
| | Peak | pH-Value | Elution pool | Peak Area |
| Condition | maximum | Elution pool | volume [mL] | [mAU * mL] |
| Excipient free | 3.65 | 4.13 | 14 | 6812 |
| 0.5M Sucrose | 3.91 | 4.25 | 18 | 6474 |
| 0.5M Trehalose | 3.83 | 4.20 | 18 | 6505 |
| 0.5M Mannitol | 3.80 | 4.22 | 18 | 6724 |
| 0.5M Sorbitol | 3.80 | 4.23 | 18 | 6729 |
| 5% PEG4000 | 3.63 | 4.11 | 14 | 6788 |

FIG. 1 shows that the addition of 5% PEG4000 causes a sharper elution peak with a significant shift to the lower pH, while elution without the use of excipient or with the use of disaccharides and polyols show broader elution peaks. According to Table 7, the volume of the elution product pool is reduced compared to the use of disaccharides and polyols and the elution product pool exhibits the lowest pH.

### Example 3: Elution Performance of Clarified Harvest mAbA on ProSep® Ultra Plus

### Protein A Chromatography Resin:

ProSep® Ultra Plus resin has a controlled pore glass matrix and recombinant native Protein A as a ligand bound to it. ProSep® Ultra Plus is from Merck (Darmstadt, Germany) and the column was packed by Repligen GmbH (Ravensburg, Germany).

TABLE 8
Column parameters for applied ProSep ® Ultra Plus resin

| | | |
|---|---|---|
| Column length | 2 | cm |
| Column inner diameter | 0.8 | cm |
| Column volume | 1 | mL |
| Mean particle diameter | 60 | μm |

| | |
|---|---|
| Base material | controlled pore glass |
| Functional group | rec. native Protein A |
| Lot # | A4SA045AQ |
| Serial # | 00227 |

Sample preparation and Protein A chromatography method were as described in Example 2.

The following results were obtained and are represented graphically as elution profiles in FIG. 2:

TABLE 9
Elution performance of clarified harvest
mAbA on ProSep ® Ultra Plus

| | pH-Value | | | |
|---|---|---|---|---|
| | Peak | pH-Value | Elution pool | Peak Area |
| Condition | maximum | Elution pool | volume [mL] | [mAU * mL] |
| Excipient free | 3.66 | 4.19 | 14 | 7300 |
| 0.5M Sucrose | 3.53 | 4.34 | 18 | 7466 |
| 0.5M Trehalose | 3.59 | 4.46 | 18 | 7373 |
| 0.5M Mannitol | 3.67 | 4.24 | 16 | 7372 |
| 0.5M Sorbitol | 3.55 | 4.24 | 18 | 7536 |
| 5% PEG4000 | 3.35 | 4.05 | 12 | 6913 |

FIG. 2 shows that the addition of 5% PEG4000 causes a sharper elution peak on ProSep® Ultra Plus, while elution without the use of excipient or with the use of disaccharides and polyols show broader elution peaks. According to Table 9, the volume of the elution product pool is the lowest compared to the samples using disaccharides, polyols or no excipient and the elution product pool exhibits the lowest pH.

### Example 4: Elution Performance of Clarified Harvest mAbA on MabSelect™ SuRe™

### Protein A Chromatography Resin:

The MabSelect™ SuRe™ resin has an agarose matrix. Immobilized onto it through thio-ether is a recombinantly produced (E. coli) tetramer of an engineered Protein A domain with a C-terminal cysteine. This resin was produced by GE Healthcare (Uppsala, Sweden) and the column was packed by Repligen GmbH (Ravensburg, Germany).

TABLE 10
Column parameters for applied Mab Select ® SuRe ™ resin

| | | |
|---|---|---|
| Column length | 2 | cm |
| Column inner diameter | 0.8 | cm |
| Column volume | 1 | mL |
| Mean particle diameter | 85 | μm |

| | |
|---|---|
| Base material | rigid, highly cross-linked agarose |
| Functional group | alkali-stabilized protein A-derived domain |
| Serial # | 00620 |

Antibody sample preparation and Protein A chromatography method were as described in Example 2.

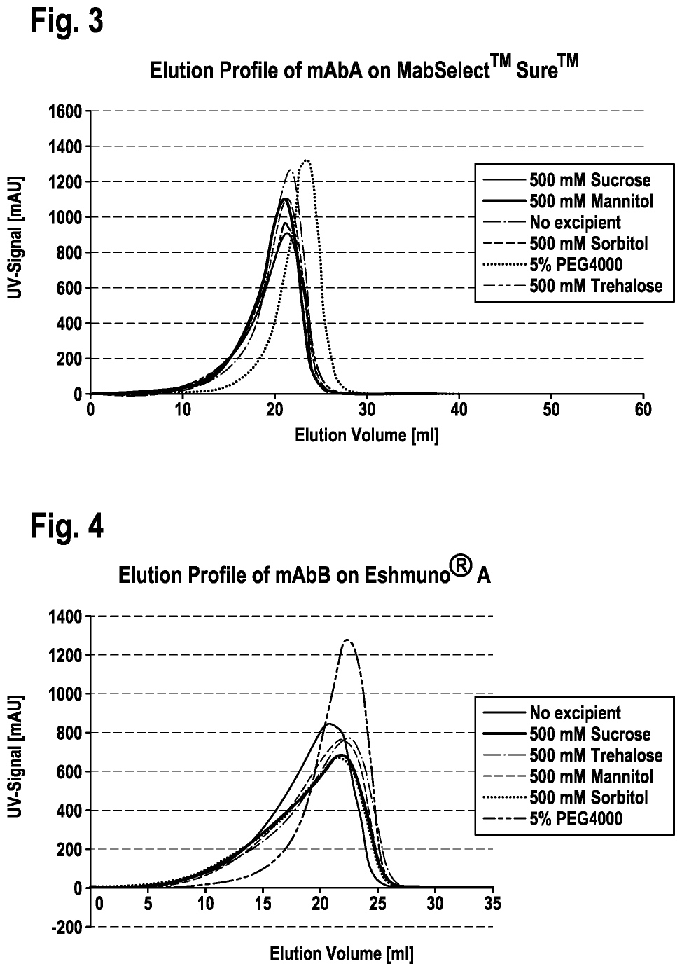

The following results were obtained and are represented graphically as elution profiles in FIG. 3:

TABLE 11
Elution performance of clarified harvest mAbA on
MabSelect ™ SuRe ™

| | pH-Value | | | |
|---|---|---|---|---|
| | Peak | pH-Value | Elution pool | Peak Area |
| Condition | maximum | Elution pool | volume [mL] | [mAU * mL] |
| Excipient free | 3.43 | 4.37 | 18 | 6664 |
| 0.5M Sucrose | 3.56 | 4.28 | 20 | 6189 |
| 0.5M Trehalose | 3.43 | 4.3 | 18 | 6367 |
| 0.5M Mannitol | 3.59 | 4.33 | 18 | 6490 |
| 0.5M Sorbitol | 3.57 | 4.36 | 18 | 6532 |
| 5% PEG4000 | 3.36 | 4.12 | 16 | 6530 |

FIG. 3 shows that the addition of 5% PEG4000 causes a sharper elution peak on MabSelect™ SuRe™ with a remarkable shift to the lower pH, while elution without the use of excipient or with the use of disaccharides and polyols show broader elution peaks. According to Table 11, the volume of the elution product pool is the lowest compared to the samples using disaccharides, polyols or no excipient and the elution product pool exhibits the lowest pH.

### Example 5: Elution Performance of Clarified Harvest mAbB on Eshmuno® A

### Antibody Sample Preparation:

The second model antibody mAbB is a monoclonal antibody (Approximately 145 kDa) produced by Merck (Darmstadt, Germany), with a pl˜7.6-8.3. It was used as clarified cell culture harvest, which was filtrated using a VacuCap® 90 PF Filter Unit with 0.8/0.2 μm Supor® membrane (Pall Corporation, NY, USA). The solution has a concentration of 1.45 mg/mL, a pH of 7.0 and a conductivity of 12.87 mS/cm.

The Protein A chromatography resin was Eshmuno® A as described in Example 2 and Protein A chromatography method was also as described in Example 2.

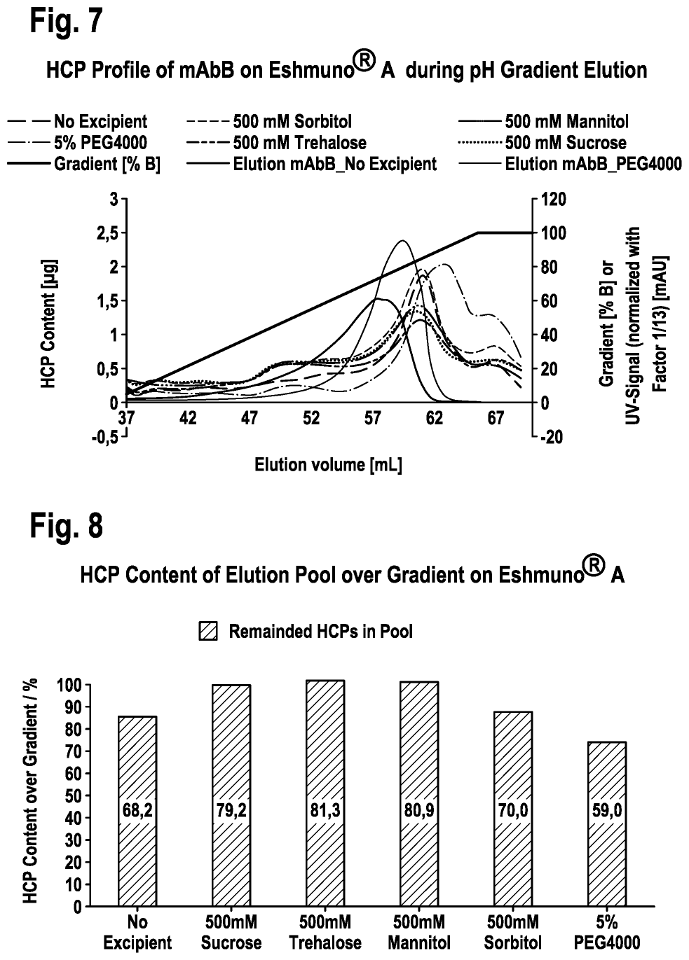

The following results were obtained and are represented graphically as elution profiles in FIG. 4, FIG. 7 and FIG. 8:

TABLE 12
Elution performance of clarified harvest mAbB on Eshmuno ® A

| | | | | | | Remained HCP |
|---|---|---|---|---|---|---|
| | | | Elution | | | in Pool over |
| | pH-Value | pH-Value | pool | | | total HCP in |
| | Peak | Elution | volume | Peak Area | Yield | Gradient |
| Condition | maximum | pool | [mL] | [mAU * mL] | [%] | [%] |
| Excipient free | 3.31 | 4.42 | 18 | 6386 | 75.5 | 68.2 |
| 0.5M Sucrose | 3.55 | 4.36 | 18 | 5977 | 70.7 | 79.2 |
| 0.5M Trehalose | 3.67 | 4.50 | 20 | 5925 | 70.0 | 81.3 |
| 0.5M Mannitol | 3.44 | 4.35 | 18 | 6241 | 73.8 | 80.9 |
| 0.5M Sorbitol | 3.54 | 4.36 | 20 | 6297 | 74.4 | 70.0 |
| 5% PEG4000 | 3.29 | 4.18 | 16 | 6749 | 79.8 | 59.0 |

FIG. 4 shows that the addition of 5% PEG4000 causes a significantly sharper elution peak on Eshmuno® A, while elution without the use of excipient or with the use of disaccharides and polyols show broad elution peaks. According to Table 12, the volume of the elution product pool is the lowest compared to the samples using disaccharides, polyols or no excipient and the elution product pool exhibits the lowest pH.

FIG. 7 shows HCP distribution over the pH gradient. HCP profiles of additive conditions with 500 mM sorbitol, mannitol, trehalose or sucrose are comparable to control condition without additive. By comparison, elution behavior of the HCP's in the presence of PEG4000 differs significantly from control and other selected additive conditions. Herein more HCPs were eluted in the rear part of the gradient in a large peak, which means HCP elution was shifted in slightly lower pH condition. This results not only in a higher purity of the elution pool (only 59% HCP vs. 68.2% remained HCP of total HCP in gradient under control condition without addition of excipient) but also higher yield (79.8% yield vs. 75.5% yield of control condition without addition of excipient).

The purple solid line in FIG. 7 represents the UV elution profile of mAbB without excipient and is only intended to illustrate the elution time of the antibody in the gradient in relation to the HCP elution. The antibody elution profiles in the presence of sorbitol, mannitol, trehalose or sucrose are similar. The UV elution profile in the presence of 5% PEG4000 is slightly shifted to higher gradient condition (lower pH condition).

HCP content of collected fractions during pH gradient from each chromatography run were analyzed and compared. FIG. 8 shows the purity of elution pool based on comparison of HCP content of elution pool from collected fractions based on UV280 collection criterion of >30 mAU, with total HCP content during pH gradient elution. Elution pool with lowest HCP content up to 59% of total HCP in gradient was achieved during chromatography run using Eshmuno® A with addition of 5% PEG4000.

### Example 6: Elution Performance of Clarified Harvest mAbB on ProSep® Ultra Plus

Protein A chromatography was carried out as described in Example 2 except that ProSep® Ultra Plus resin as described in Example 3 was used as the Protein A chromatography resin the and mAbB as described in Example 5 was used as the antibody.

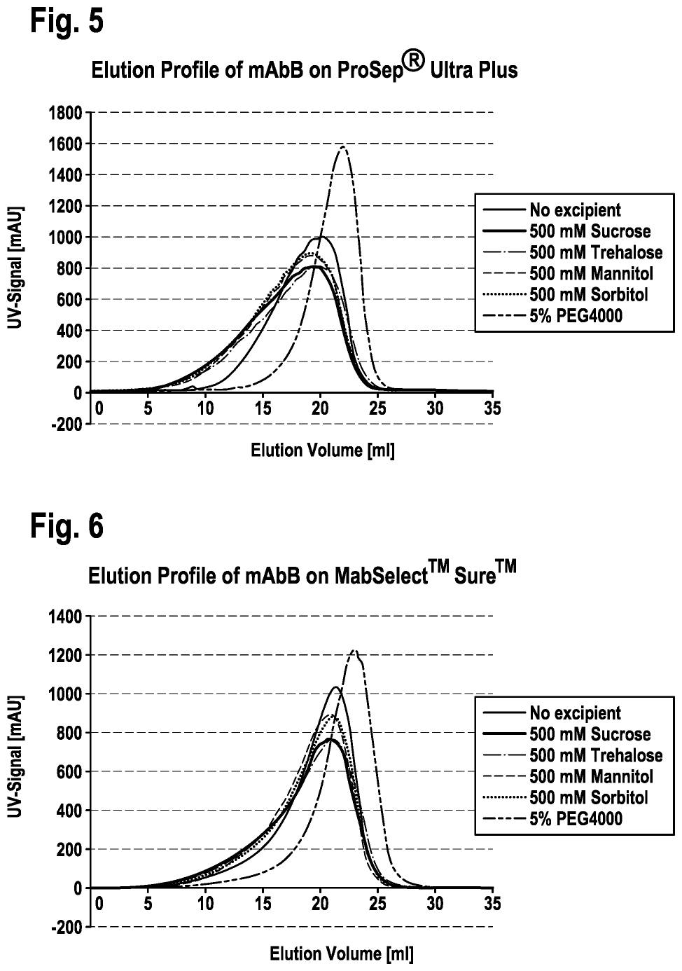

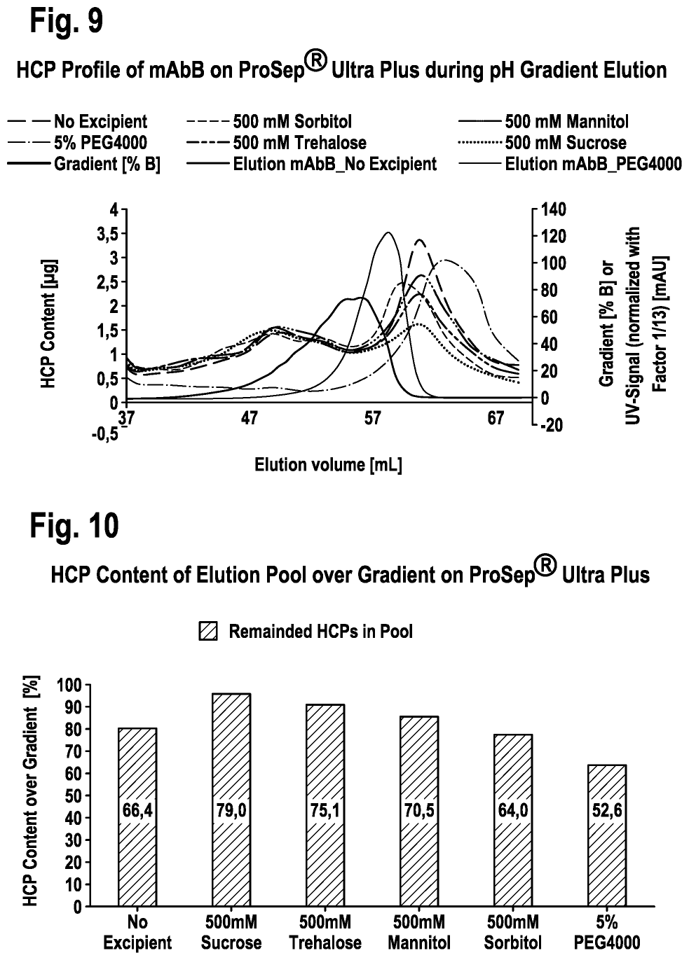

The following results were obtained and are represented graphically as elution profiles in FIG. 5, FIG. 9 and FIG. 10:

TABLE 13
Elution performance of clarified harvest mAbB on ProSep ® Ultra Plus

| | | | | | | Remained HCP |
|---|---|---|---|---|---|---|
| | | | Elution | | | in Pool over |
| | pH-Value | pH-Value | pool | | | total HCP in |
| | Peak | Elution | volume | Peak Area | Yield | Gradient |
| Condition | maximum | pool | [mL] | [mAU * mL] | [%] | [%] |
| Excipient free | 3.65 | 4.17 | 16 | 7020 | 83.0 | 66.4 |
| 0.5M Sucrose | 3.84 | 4.49 | 20 | 7374 | 87.2 | 79.0 |
| 0.5M Trehalose | 3.73 | 4.54 | 20 | 7459 | 88.2 | 75.1 |
| 0.5M Mannitol | 3.80 | 4.48 | 18 | 7477 | 88.4 | 70.5 |
| 0.5M Sorbitol | 3.71 | 4.42 | 20 | 7605 | 89.9 | 64.0 |
| 5% PEG4000 | 3.40 | 4.18 | 14 | 7298 | 86.3 | 52.6 |

FIG. 5 shows that the addition of 5% PEG4000 causes a significantly sharper elution peak on ProSep® Ultra Plus with a remarkable shift to the lower pH, while elution without the use of excipient or with the use of disaccharides and polyols show broad elution peaks. According to Table 13, the volume of the elution product pool is the lowest compared to the samples using disaccharides, polyols or no excipient.

FIG. 9 shows HCP distribution over the pH gradient. HCP profiles of additive conditions with 500 mM sorbitol, mannitol, trehalose or sucrose are comparable to control condition without additive. By comparison, elution behavior of the HCP's in the presence of PEG4000 differs significantly from control and other selected additive conditions. Herein more HCPs were eluted in the rear part of the gradient in a large peak, which means HCP elution was shifted in slightly lower pH condition. This results not only in a higher purity elution pool (only 52.6% remained HCP vs. 66.4% remained HCP of total HCP in gradient in control condition without addition of excipient) but also higher yield (86.3% yield vs. 83% yield of control condition without addition of excipient).

The purple solid line in FIG. 9 represents the UV elution profile of mAbB without excipient and is only intended to illustrate the elution time of the antibody in the gradient in relation to the HCP elution. The antibody elution profiles in the presence of sorbitol, mannitol, trehalose or sucrose are similar. The UV elution profile in the presence of 5% PEG4000 is slightly shifted to higher gradient condition (lower pH condition).

HCP content of collected fractions during pH gradient from each chromatography run were analyzed and compared. FIG. 10 shows the purity of elution pool based on comparison of HCP content of elution pool from collected fractions based on UV280 collection criterion of >30 mAU, with total HCP content during pH gradient elution. Elution pool with lowest HCP content up to 52.6% of total HCP in gradient was achieved during chromatography run using ProSep® Ultra Plus with addition of 5% PEG4000.

### Example 7: Elution Performance of Clarified Harvest mAbB on MabSelect™ SuRe™

Protein A chromatography was carried out as described in Example 2 except that MabSelect™ SuRe™ resin as described in Example 4 was used as the Protein A chromatography resin the and mAbB as described in Example 5 was used as the antibody.

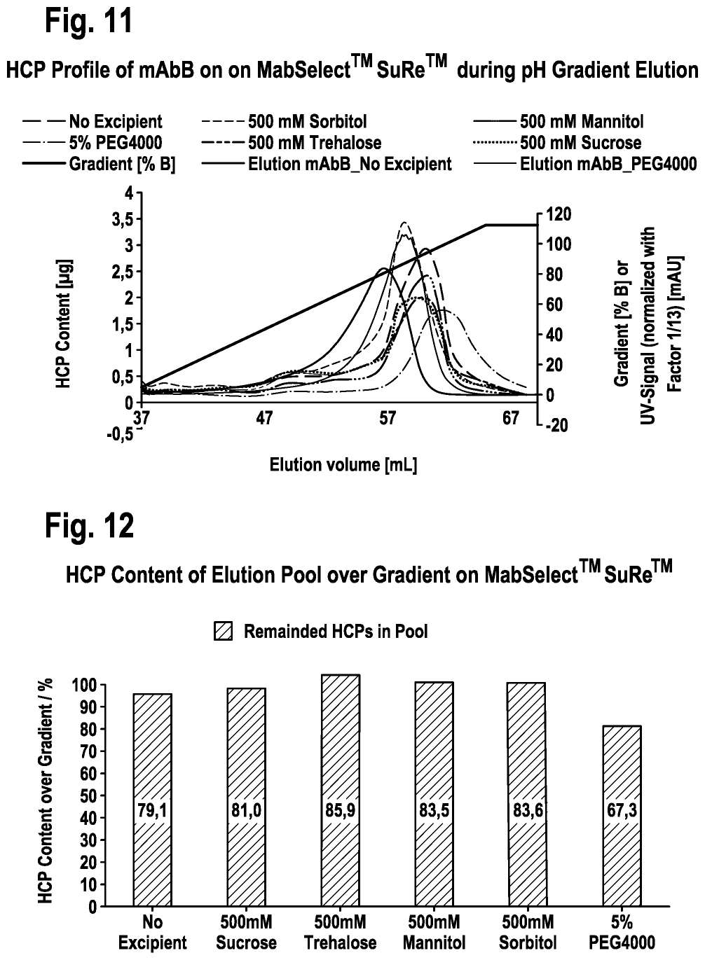

The following results were obtained and are represented graphically as elution profiles in FIG. 6, FIG. 11 and FIG. 12:

TABLE 14
Elution performance of clarified harvest mAbB on MabSelect ™ SuRe ™

| | | | | | | Remained HCP |
|---|---|---|---|---|---|---|
| | | | Elution | | | in Pool over |
| | pH-Value | pH-Value | pool | | | total HCP in |
| | Peak | Elution | volume | Peak Area | Yield | Gradient |
| Condition | maximum | pool | [mL] | [mAU * mL] | [%] | [%] |
| Excipient free | 3.56 | 4.39 | 18 | 6477 | 76.6 | 79.1 |
| 0.5M Sucrose | 3.71 | 4.40 | 20 | 6107 | 72.2 | 81.0 |
| 0.5M Trehalose | 3.58 | 4.35 | 20 | 6105 | 72.2 | 85.9 |
| 0.5M Mannitol | 3.57 | 4.42 | 18 | 6415 | 75.8 | 83.5 |
| 0.5M Sorbitol | 3.63 | 4.39 | 20 | 6379 | 75.4 | 83.6 |
| 5% PEG4000 | 3.40 | 4.15 | 18 | 6572 | 77.7 | 67.3 |

FIG. 7 shows that the addition of 5% PEG4000 causes a sharper elution peak on MabSelect™ SuRe™ with a remarkable shift to the lower pH, while elution without the use of excipient or with the use of disaccharides and polyols show broader elution peaks. According to Table 14, the pH of the elution product pool is the lowest compared to the samples using disaccharides, polyols or no excipient.

FIG. 11 shows HCP distribution over the pH gradient. HCP profiles of additive conditions with 500 mM sorbitol, mannitol, trehalose or sucrose are comparable to control condition without additive. By comparison, elution behavior of the HCP's in the presence of PEG4000 differs significantly from control and other selected additive conditions. Herein more HCPs were eluted in the rear part of the gradient in a large peak, which means HCP elution was shifted in slightly lower pH condition. This results not only in a higher purity elution pool (only 67.3% remained HCP vs. 79.1% remained HCP of total HCP in gradient in control condition without addition of excipient) but also higher yield (77.7% yield vs. 76.6% yield of control condition without addition of excipient).

The purple solid line in FIG. 11 represents the UV elution profile of mAbB without excipient and is only intended to illustrate the elution time of the antibody in the gradient in relation to the HCP elution. The antibody elution profiles in the presence of sorbitol, mannitol, trehalose or sucrose are similar. The UV elution profile in the presence of 5% PEG4000 is slightly shifted to higher gradient condition (lower pH condition).

HCP content of collected fractions during pH gradient from each chromatography run were analyzed and compared. FIG. 12 shows the purity of elution pool based on comparison of HCP content of elution pool from collected fractions based on UV280 collection criterion of >30 mAU, with total HCP content during pH gradient elution. Elution pool with lowest HCP content up to 67.3% of total HCP in gradient was achieved during chromatography run using MabSelect™ SuRe™ with addition of 5% PEG4000.

1. Method of separating a monoclonal antibody from a Protein A affinity chromatography column to which the monoclonal antibody is bound by use of a reduced volume of eluent comprising the steps of:
a) bringing the loaded affinity chromatography column into contact with an elution buffer comprising a poly (ethylene glycol) polymer and inducing a sharp, narrow elution peak,
b) collecting one or more of the eluted fractions containing the monoclonal antibody obtained from step (a),
c) potentially combining the fractions obtained from step (b) to form an elution product pool,
whereby
an improved purity of the desired monoclonal antibody is achieved and about 9% and more of HCP is separated off and an increase in yield by more than 4% is achieved.

2. The method according to claim 1, wherein the elution buffer has a concentration of poly (ethylene glycol) polymer from about 2% to 15% by weight.

3. The method according to claim 2, wherein the elution buffer has a concentration of poly (ethylene glycol) polymer from about 5% to 10% by weight.

4. The method according to claim 1, wherein the poly (ethylene glycol) polymer has an average molecular weight from 1,000 g/mol to 10,000 g/mol.

5. The method according to claim 1, wherein the poly (ethylene glycol) polymer has an average molecular weight from 3,000 g/mol to 6,000 g/mol.

6. The method according to claim 1, wherein the elution buffer is a citrate buffer.

7. The method according to claim 1, wherein the elution step (a) comprises contacting the affinity chromatography column with the elution buffer using an elution buffer gradient from pH 5.5 to pH 2.75.

8. The method according to claim 1, wherein the elution product pool has a pH in the range from about 3.9 to about 4.2.

9. The method according to claim 1, characterized in that the demand for eluent is reduced by about at least 11%.
